(19) United States
(12) Patent Application Publication
(10) Pub. No.: US 2007/0293871 A1
(43) Pub. Date: Dec. 20, 2007

(54) Osteotome and components thereof

(76) Inventor: Andrew Ackermann, Sandton (ZA)

Correspondence Address: MARSHALL & MELHORN, FOUR SEAGATE, EIGHT FLOOR, TOLEDO, OH, 43604, US

(21) Appl. No.: 11/786,942

(22) Filed: Apr. 13, 2007

(30) Foreign Application Priority Data
Apr. 13, 2006 (ZA) 2006/03017

(51) Int. Cl. A61B 17/58; A61B 17/00; B21J 15/10

(52) U.S. Cl. 606/99; 227/52; 606/84

(57) ABSTRACT

An osteotome includes a penetration tool which is to be driven into a bone and a separate, elongate driving tool for use in driving the penetration tool into the bone. The penetration tool has a tapered penetration end and an opposite end including at least one curved formation. The driving tool has an operative end including another curved formation which is shaped to engage the curved formation at the said opposite end of the penetration tool. With the curved formation at the operative end of the driving tool engaged with the curved formation at the said opposite end of the penetration tool, the driving tool can be used to transmit a driving force to the penetration tool to drive the penetration tool into the bone. The complemental curvatures of the formations enable the driving tool to be applied to the penetration tool at different angles, facilitating access to different regions of the mouth. The invention encompasses the penetration tool and the driving tool per se.

### BACKGROUND OF THE INVENTION

This invention relates to an osteotome and to components of the osteotome.

An osteotome is used, as an alternative to drilling, to form holes in bone. In a typical application, an osteotome may be used to form a hole in the bone of the maxilla or mandible for the purposes of anchoring a dental implant. A conventional osteotome used in this application consists of a rigid, elongate tool with a sharp end. In use the osteotome is held at the appropriate orientation relative to the bone and is then driven into the bone to form the hole. This is typically achieved either by applying hammer blows to the free end of the osteotome or by manually working the osteotome into the bone. It is also conventional practice to initiate the procedure with a pilot hole of small diameter using an osteotome with a thin working end and then progressively increase the diameter of the hole using other osteotomes with working ends of progressively increasing thickness.

Compared to drilling, advantages of using an osteotome to form the hole include the fact that bone is not removed and that the bone surrounding the hole which is formed is compressed and is accordingly better able to anchor the dental implant. However a disadvantage which arises with conventional osteotomes is the fact that space constraints and jaw geometry can make it difficult to align the tool properly and drive it into the bone at the correct orientation, particularly when the hole is to be formed towards the back of the mouth.

An object of this invention is to provide an osteotome which addresses this problem.

### SUMMARY OF THE INVENTION

An osteotome according to the present invention comprises a penetration tool which is to be driven into a bone and a separate, elongate driving tool for use in driving the penetration tool into the bone, the penetration tool having a tapered penetration end and an opposite end including at least one curved formation and the driving tool having an operative end including another curved formation which is shaped to engage the curved formation at the said opposite end of the penetration tool whereby, with the curved formation at the operative end of the driving tool engaged with the curved formation at the said opposite end of the penetration tool, the driving tool can be used to transmit a driving force to the penetration tool to drive the penetration tool into the bone.

Engagement between the respective curved formations enables the driving tool to be engaged with the penetration tool at any one of a variety of different angles.

In the preferred embodiment, the said opposite end of the penetration tool includes a plurality of angularly spaced, curved formations which are selectively engagable by the curved formation at the operative end of the driving tool. The said opposite end of the penetration tool may also include an axial projection and a further curved formation in an end of the projection. The curved formations at the said opposite end of the penetration tool are preferably spherically curved recesses, and the curved formation at the operative end of the driving tool is a spherically curved convexity complemental to the spherically curved recesses.

Conveniently, the penetration tool has a shape generally matching that of an implant which is to be anchored in a hole formed in use in the bone by the penetration tool. To this end, the preferred penetration tool includes a cylindrical portion and a tapered portion extending from the cylindrical portion to the penetration end, with the tapered and cylindrical portions defining a shape corresponding to that of a tapered, threaded implant which is to be anchored in the hole. In practice, the combined length of the tapered and cylindrical portions of the penetration tool may be selected to be substantially the same as that of the implant. The lateral dimensions of the tapered and cylindrical portions of the penetration tool may be selected to be slightly less than an external diameter of threads on the implant.

The driving tool may for instance comprise an elongate, straight or cranked body with the operative end located at one end of the body.

The invention extends to an osteotome penetration tool, the penetration tool including a tapered penetration end, a cylindrical portion and a tapered portion extending from the cylindrical portion to the penetration end, whereby the penetration tool has a shape generally matching that of an implant which is to be anchored in a hole formed in use in a bone by the penetration tool, the cylindrical portion of the penetration tool defining an opposite end having at least one curved recess therein which is engagable by an operative end of a driving tool of the osteotome which can used to drive the penetration tool into the bone.

The invention also extends to an osteotome driving tool for use in driving an osteotome penetration tool into a bone, the driving tool comprising an elongate body having an operative end including a convexly curved tip shaped to engage a complementally curved recess at an end of the penetration tool, whereby the driving tool can be used to drive the penetration tool into the bone with the driving tool arranged at any one of a variety of different orientations of the driving tool relative to the penetration tool.

### BRIEF DESCRIPTION OF THE DRAWINGS

The invention will now be described in more detail, by way of example only, with reference to the accompanying drawings.

In the drawings:

FIG. 1 shows a perspective view of an osteotome according to the invention;

FIG. 2 shows a side view of the penetration tool of the osteotome;

FIG. 3 shows an end view, on the arrow 3 in FIG. 2, of the penetration tool;

FIG. 4 shows a side view of the driving tool of the osteotome;

FIG. 5 illustrates the operation of the osteotome;

FIG. 6 diagrammatically illustrates a portion of a maxilla or mandible and sockets left therein after removal of a molar tooth therefrom;

FIG. 7 shows a side view of a dental implant which is to be anchored in a hole formed in use by the osteotome; and

FIG. 8 shows a side view of another embodiment of a driving tool in accordance with the invention.

### DESCRIPTION OF PREFERRED EMBODIMENTS

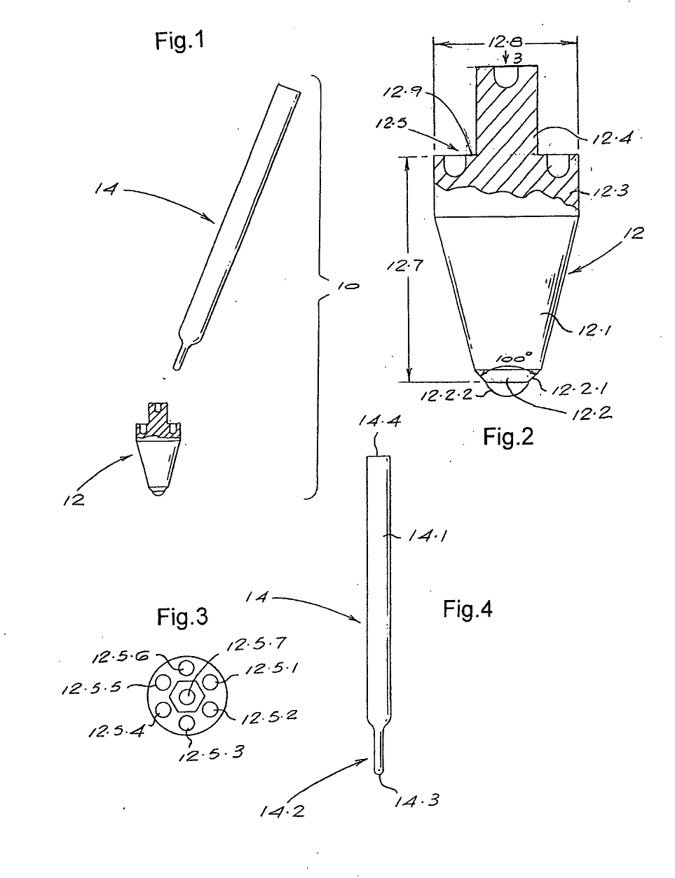

The osteotome 10 seen in FIG. 1 consists of a penetration tool 12 and a driving tool 14.

Referring also to FIGS. 2 and 3, the penetration tool 12 has a tapered portion 12.1 leading to a penetration end 12.2 and also includes a cylindrical portion 12.3 remote from the penetration end. Extending axially from the cylindrical portion 12.3 is a projection 12.4.

As shown, the penetration end 12.2 includes a tapered portion 12.2.1 defining an included angle of 100° and a guide portion 12.2.2. In this embodiment of the invention the penetration tool 12 includes a curved formation, generally indicated with the reference numeral 12.5 comprising six spherically curved recesses or concavities 12.5.1, 12.5.2, 12.5.3, . . . , 12.5.6 formed in the end of the cylindrical portion 12.3. The penetration tool includes a further curved formation provided by a spherically curved recess or concavity 12.5.7 formed in the end of the projection 12.4.

Referring also to FIG. 4, the driving tool 14 has a straight body 14.1 of round cross-section terminating at an operative end 14.2 of smaller round cross-section and formed with a spherically curved convexity 14.3.

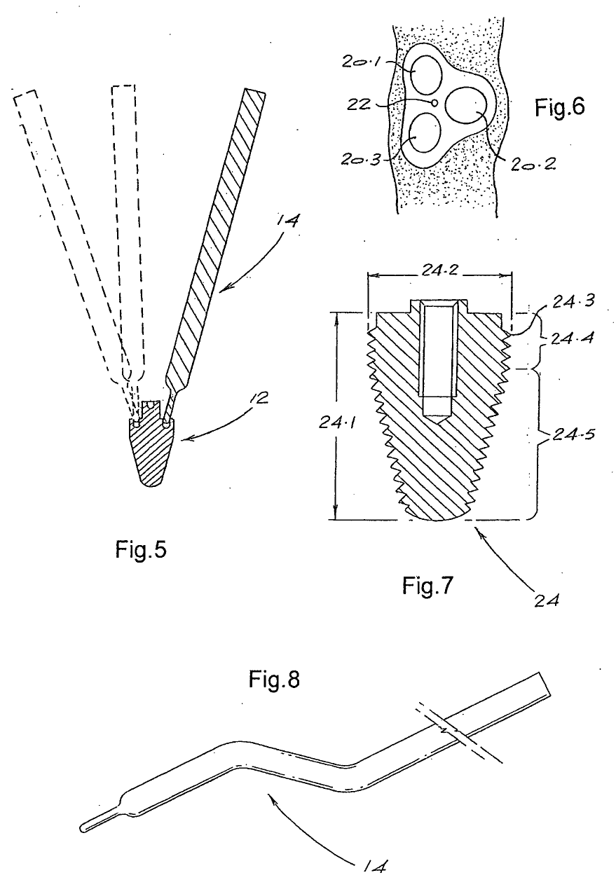

The radius of curvature defining the convexity 14.3 is the same as the radius of curvature defining the concavities 12.5.1, 12.5.2, . . . , 12.5.7, i.e. the convexity and concavities are complemental. The complemental nature of the curvatures allows the driving tool to be engaged with the penetration tool at a variety of angles, as illustrated by the full and broken lines in FIG. 5.

FIG. 6 provides a diagrammatic representation of three root sockets 20.1, 20.2 and 20.3 left by a molar tooth which has been removed from a maxillary bone. A pilot hole 22 has been drilled in the bone at a location between the three extraction sockets.

The osteotome 10 can now be used to form a hole in the maxillary or mandibular bone, for anchorage of a dental implant in the position of the removed molar tooth. In use, the pointed penetration end 12.2 of the penetration tool 12 is placed against the bone at the appropriate orientation, with the guide portion 12.2.2 being located inside the pilot hole 22. The operative end 14.2 of the driving tool 14 is then fitted into one of the concavities 12.5.1, 12.5.2, . . . , 12.5.7 at an appropriate orientation, whereafter the driving tool is used to drive the penetration tool into the bone.

This may for instance be achieved by hammering on its end 14.4 or alternatively by manually working the tool such that it transmits a driving force to the penetration tool.

It will be appreciated that if the driving tool is not perfectly aligned with the penetration tool, only a component of the force applied to the driving tool is transmitted to the penetration tool to drive it in the desired direction into the bone. Nevertheless it is anticipated, given the softness of the bone and particularly the bone of the maxilla, that the driving force which is applied to the penetration tool will be sufficient to drive it into the bone.

The ability of the driving tool to transmit a driving force to the penetration tool at a variety of angles is advantageous in that it enables the osteotome to function even in relatively inaccessible places in the mouth, for instance at the back of the mouth. An additional advantage of this feature is the fact that the driving tool can be reorientated as necessary during the driving procedure such that the lateral force which it applies to the penetration tool can be used to steer it in the correct direction into the bone. This may for instance be necessary if it is found that the penetration tool is wandering from the correct alignment as it penetrates the bone.

An additional advantage of the osteotome arises from the fact that the penetration tool 12 has a shape generally matching that of the implant which is to be anchored in the hole which is formed in the bone. FIG. 7 shows a threaded, tapered molar implant 24 which is to be anchored in the bone and for which the penetration tool 12 is specifically designed.

The molar implant 24 is described in detail in the specification of the applicant's co-pending international patent application PCT/IB2006/001373 filed on 26 May 2006, in particular with reference to FIG. 3 of that document. The implant has a length dimension 24.1 that can vary between 7 mm and 15 mm and the lateral dimension, i.e. diameter 24.2, measured to the lateral extremities of the threads 24.3, is about 8.0 mm. The implant has tapered and cylindrical portions 24.4 and 24.5 respectively.

As indicated above, the shape of the penetration tool 12 generally matches that of the implant. In the illustrated case, the length dimension 24.1 of the implant may be 11 mm, matching the length dimension 12.7 of the penetration tool. The tapered and cylindrical portions 12.1 and 12.3 of the penetration tool may have the same lengths as the tapered and cylindrical portions 24.5 and 24.4 of the implant, and the taper angles of the tapered portions may also be the same. The diameter 12.8 of the penetration tool may be about 7.3 mm, very slightly less than the dimension 24.2 of the implant.

The configuration of the penetration tool is accordingly such that, when driven fully into the bone to the level of the surface 12.9, it forms a hole in the bone having a shape similar to that of the implant which is to be anchored therein. The slightly smaller lateral dimensions of the penetration tool, and accordingly of the hole which is formed, enables the thread of the implant to engage the bone securely when the implant is screwed into the hole.

The advantage of matching the shapes of the penetration tool and implant is that the penetration tool can, in a single step, form a hole which is optimally dimensioned to receive and anchor the implant.

It will be understood that a variety of different driving tools may be used to drive the penetration tool. At each stage of the driving procedure a driving tool can be selected which is best suited to the particular circumstances. For instance space restrictions may make it appropriate to use a relatively short driving tool at the commencement of the driving procedure while a longer tool may be used later on when the penetration tool has been driven some way into the bone and more space is available. Driving tools which are not straight may also be used where the jaw geometry makes this appropriate. This is exemplified in FIG. 8 which shows a driving tool 14 with a cranked shape.

It is within the scope of the invention for the penetration and driving tools to have cooperating formations which are not spherically curved. It is also within the scope of the invention for the penetration tool to have convexities rather than concavities as described above, and for the driving tool correspondingly to have a concavity rather than a convexity.

The projection 12.4 provides purchase for an extraction tool (not shown) which can be used to pull the penetration tool out of the hole once the latter has been formed. The projection has a hexagonal cross-section to facilitate rotation of the tool.

The illustrated penetration tool and driving tools are made of stainless steel, but other suitable materials may also be used.

What is claimed is:

1. An osteotome comprising a penetration tool which is to be driven into a bone and a separate, elongate driving tool for use in driving the penetration tool into the bone, the penetration tool having a tapered penetration end and an opposite end including at least one curved formation and the driving tool having an operative end including another curved formation which is shaped to engage the curved formation at the said opposite end of the penetration tool whereby, with the curved formation at the operative end of the driving tool engaged with the curved formation at the said opposite end of the penetration tool, the driving tool can be used to transmit a driving force to the penetration tool to drive the penetration tool into the bone.

2. An osteotome according to claim 1 wherein the said opposite end of the penetration tool includes a plurality of angularly spaced, curved formations which are selectively engagable by the curved formation at the operative end of the driving tool.

3. An osteotome according to claim 2 wherein the said opposite end of the penetration tool includes an axial projection and a further curved formation in an end of the projection.

4. An osteotome according claim 3 wherein curved formations at the said opposite end of the penetration tool are spherically curved recesses.

5. An osteotome according to claim 4 wherein the curved formation at the operative end of the driving tool is a spherically curved convexity complemental to the spherically curved recesses.

6. An osteotome according to claim 1 wherein the penetration tool has a shape generally matching that of an implant which is to be anchored in a hole formed in use in the bone by the penetration tool.

7. An osteotome according to claim 6 wherein the penetration tool includes a cylindrical portion and a tapered portion extending from the cylindrical portion to the penetration end.

8. An osteotome according to claim 7 wherein the tapered and cylindrical portions of the penetration tool define a shape corresponding to that of a tapered, threaded implant which is to be anchored in the hole.

9. An osteotome according to claim 8 wherein a combined length of the tapered and cylindrical portions of the penetration tool is selected to be substantially the same as that of the implant.

10. An osteotome according to claim 9 wherein lateral dimensions of the tapered and cylindrical portions of the penetration tool are selected to be slightly less than an external diameter of threads on the implant.

11. An osteotome according to claim 7 wherein the penetration tool includes a projection extending axially from the cylindrical portion in a direction opposite to that in which the tapered portion extends from the cylindrical portion, the projection having a curved recess in an end surface thereof remote from the tapered portion.

12. An osteotome according to claim 1 wherein the driving tool comprises an elongate, straight body with the operative end located at one end of the body.

13. An osteotome according to claim 1 wherein the driving tool comprises an elongate, cranked body with the operative end located at one end of the body.

14. An osteotome penetration tool, the penetration tool including a tapered penetration end, a cylindrical portion and a tapered portion extending from the cylindrical portion to the penetration end, whereby the penetration tool has a shape generally matching that of an implant which is to be anchored in a hole formed in use in a bone by the penetration tool, the cylindrical portion of the penetration tool defining an opposite end having at least one curved recess therein which is engagable by an operative end of a driving tool of the osteotome which can used to drive the penetration tool into the bone.

15. An osteotome penetration tool according to claim 14 wherein the penetration tool includes a projection extending axially from the cylindrical portion in a direction opposite to that in which the tapered portion extends from the cylindrical portion, the projection having a curved recess in an end surface thereof remote from the tapered portion.

16. An osteotome penetration tool according to claim 15 wherein the diameter of the cylindrical portion is about 7.3 mm.

17. An osteotome penetration tool according to claim 16 wherein the combined axial length of the cylindrical and tapered portions is about 11 mm.

18. An osteotome driving tool for use in driving an osteotome penetration tool into a bone, the driving tool comprising an elongate body having an operative end including a convexly curved tip shaped to engage a complementally curved recess at an end of the penetration tool, whereby the driving tool can be used to drive the penetration tool into the bone with the driving tool arranged at any one of a variety of different orientations of the driving tool relative to the penetration tool.

19. A driving tool according to claim 18 wherein the body is straight.

20. A driving tool according to claim 18 wherein the body is cranked.
